(19) United States
(12) Patent Application Publication
(10) Pub. No.: US 2006/0024353 A1
(43) Pub. Date: Feb. 2, 2006

(54) Novel porous film-forming granules, process for their preparation and application in the film coating of tablets and sweets

(76) Inventors: Gerard Trouve, Castres (FR); David Girard, Toulouse (FR); Michel Malandain, Fourqueux (FR)

Correspondence Address: Linda K. Russell;Suite 1800, 2700 Post Oak Blvd., Houston, TX, 77056, US

(21) Appl. No.: 11/030,827

(22) Filed: Jan. 7, 2005

(30) Foreign Application Priority Data
Aug. 1, 2004 (FR) 0450049

(51) Int. Cl. A61K 9/68; A61K 31/716

(52) U.S. Cl. 424/440; 514/57

(57) ABSTRACT

A film forming composition and a method for its use. The composition is in the form of somewhat porous granules and has a bulk density of less than about 0.5 g/cm³. The composition contains a film-forming polymer, a plasticizer and an inert filler.

A subject-matter of the present invention is film-forming compositions intended for the coloured coating of solid ingestible forms and more particularly of pharmaceutical tablets, sweets or sugar-coated tablets, their process of preparation and a process for coating and colouring the said forms.

The film coating of tablets by coloured polymer films is commonly used in the pharmaceutical industry. This process comprises the preparation of a dispersion, preferably an aqueous dispersion, comprising a film-forming polymer, a colouring system, preferably pigments or lakes, and optionally a plasticizer, fillers and/or technological additives, and then the spraying of the dispersion over the tablets moving in a perforated rotating pan or in a fluidized bed. A stream of hot air, entering at a temperature generally of greater than or equal to 40° C., provides for the drying of the sprayed dispersion and brings about the coalescence of the film around the tablets. The deposited layer has a thickness of a few tens of microns; it is opaque and homogeneously coloured.

In order to remain at an acceptable viscosity level, generally of less than 1000 mPa·s, compatible with passage through the spray nozzles, the dispersion used is not very concentrated; it generally comprises only from 10 to 20% by weight of solids.

The components used to prepare these films have to conform to the regulations in force in the pharmaceutical, dietary or food field. This is why the polymers are generally chosen from cellulose derivatives, such as hydroxypropylmethyl celluloses (HPMC) or methyl celluloses (MC), starch derivatives, such as, for example, film-forming dextrins or maltodextrins, polyvinyl alcohols or acrylic polymers. The most widely used plasticizers in the pharmaceutical field are polyethylene glycols (PEG) with molecular weights of between approximately 300 and approximately 8000. On the other hand, they are not allowed for food applications, for which glycerol is often used as example of plasticizers. As filler, there is cellulose, lactose, sorbitol, mannitol, xylitol, silica or talc. The technological additives include, for example, surfactants, such as polysorbates, which act as wetting agents and dispersing agents.

Use may also advantageously be made of ready-to-use compositions comprising the mixtures of these various constituents in the form of powders, of granules or of extrudates. The ready-to-use compositions have several advantages:
the handling, the storage or the monitoring of one and only one product;
better reproducibility of the colours and performance;
easier dispersability.

This final point is particularly important as the dispersion of powders introduced one by one into an aqueous solvent is often a problematic operation to carry out due to the formation of foam, of lumps or of agglomerates, sometimes described as “fish eyes”, which are very difficult to disperse when they are formed. Furthermore, complete dispersion often requires a stirring time which is often very long.

The dry edible coating composition in the form of powders which is disclosed in the French patent application published under the number FR 2 470 598 does not completely solve this problem as phenomena of segregation are observed during its use which result in heterogeneous compositions in the containers of products.

The more stable compositions in the form of granules or extrudates disclosed in the patent applications published under the numbers FR 2 548 675, FR 2 660 317 and WO 99/24020 do not exhibit this disadvantage as they are agglomerates of several tens of to several thousand particles of matter, initially separated, which can be identical or different in nature and which were assembled by means of a liquid binder by a wet granulation process in a mixer-granulator or in a fluidized bed or by an extrusion process, which processes are described, for example, in the Kirk-Othmer encyclopaedia (3rd edition, volume 17, page 281). It is observed that the dimensions of the granules or extrudates obtained are of the order of 0.1 to 2 mm, whereas the starting particles of powder have dimensions of the order of a few tens of microns. Furthermore, these granules or these extrudates are particularly easy to handle as they flow freely and do not generate dust during their use. However, such ready-to-use forms are fairly difficult and expensive to manufacture. This is because the manufacturing process, as disclosed, for example, in the patents published under numbers FR 2 548 675 and FR 2 660 317, comprises the following successive stages:
wetting the powders and pigments with a binder solution, in order to obtain a wet mass comprising from 30 to 60% of water,
drying in an oven or in a fluidized bed, and
optionally grading.

Furthermore, this process results in cleaning operations on the numerous items of equipment employed during the various stages, which are particularly difficult when coloured compositions are prepared.

Finally, the ready-to-use compositions in the form of dry powders, of granules or extrudates are easily dispersed in water, provided that they are gradually run in with vigorous stirring with a strong shearing effect, for example using a deflocculating paddle. They make it possible to prepare the aqueous dispersion to be sprayed over the tablets in a time which is shorter than when all the ingredients necessary for the film coating are introduced one by one but which is still, however, relatively long as it is of the order of 30 to 45 minutes.

This is why the inventors have sought to develop novel granules and their use in a simple process for the preparation of ready-to-use film-forming compositions in the form of stable granules which do not exhibit phenomena of segregation on storage and which are rapidly dispersed in water.

According to a first aspect, a subject-matter of the invention is a film-forming composition in the form of granules with a non-zero porosity π, characterized in that it comprises:
from 30% to 100% by weight of a film-forming polymer or of a blend of several of these polymers,
from 0% to 30% by weight of a plasticizer or of a mixture of plasticizers, and
from 0% to 70% by weight of one or more inert fillers,
   and in that its bulk density g is less than 0.5 g/cm³.

The term “bulk density μ” denotes the ratio M/V, in which M represents the weight of the material and V its bulk volume. The bulk density g is determined according to the experimental protocol 2-9-15 of the European Pharmacopoeia. μ is preferably greater than or equal to 0.2 g/cm³.

The term “porosity π” denotes the ratio of the volume of the spaces of a material to its total volume. π is equal to 0 for a dense product and tends towards 1 when the material becomes increasingly porous. It corresponds to the equation: π=(a−μ)/a, in which a represents the true density of the material.

The term “film-forming polymer” denotes mainly edible polymers chosen from cellulose derivatives, such as, for example, hydroxypropylmethyl celluloses (HPMC), ethyl celluloses (EC), methyl celluloses (MC), carboxy-methyl celluloses, hydroxypropyl celluloses (HPC) or cellulose acetates or phthalates, carrageenans, sodium, potassium or ammonium alginates, or film-forming modified starch derivatives, such as, for example, dextrins, maltodextrins, guar gum, gum tragacanth, gum arabic or xanthan gum, and, for non-food applications, polyvinylpyrrolidones, polyvinyl alcohols or acrylic polymers. Use may also be made of polymers already available commercially in the form of granules, such as, for example, granulated HPMC, known under the name of Pharmacoat™ G. Use may also be made of blends of different polymers, such as, for example, HPMC/EC blends.

The composition as defined above more particularly comprises from 50% to 100% by weight of film-forming polymers and very particularly 50% to 90% by weight of film-forming polymers.

The term “plasticizer” is understood to mean mainly either soluble plasticizers, such as polyols in general and more particularly sorbitol, mannitol, xylitol, glycerol, sucrose, polyethylene glycols or propylene glycol, or plasticizers of low solubility in water, such as those comprising an aliphatic chain comprising at least 12 carbon atoms, for example stearic acid, stearic acid salts, such as magnesium stearate or aluminium stearate, polyethoxylated stearic acid, fatty acid monoglycerides, fatty acid diglycerides and their derivatives esterified by acetic acid, tartaric acid or lactic acid, esters of fatty acids and of propylene glycol, esters of fatty acids and of sorbitol, esters of fatty acids and of sorbitan, esters of fatty acids and of mannitol, esters of fatty acids and of mannitan, or also certain sucrose esters, sucroglycerides or polyglycerol esters, in particular those characterized by an HLB number of less than 7.

The composition as defined above more particularly comprises from 0% to 20% by weight of a plasticizer or of a mixture of plasticizers.

The term “inert filler” mainly denotes microcrystalline cellulose, lactose, talc or silica.

The composition as defined above more particularly comprises from 0% to 50% by weight of one or more inert fillers and very particularly from 10% to 50% by weight of one or more inert fillers.

The composition as defined above has a residual water content of less than 10% by weight, preferably of less than 6% by weight.

The composition as defined above generally has a particle size distribution such that at most 25% of its total weight has a particle size of less than 500 μm and at most 15% of its total weight has a particle size of greater than 2000 μm, and a mean diameter of approximately 800 μm, preferably of between 600 and 1000 μm. In a preferred version, the particle size distribution of the composition is such that at most 20% of its total weight has a particle size of less than 500 μm and at most 15% of its total weight has a of particle size of greater than 1500 μm.

Another subject-matter of the invention is a process for the preparation of the composition as defined above, characterized in that the film-forming polymer or the blend of several of these polymers, optionally the plasticizer and/or optionally the inert filler are homogenized in a fluidized bed by blowing with air and then in that water is gradually sprayed therein until a wet mass is formed, which is simultaneously or subsequently dried by blowing with hot air, preferably at a temperature of between 70° C. and 100° C.

As is shown in the experimental examples expanded upon later in the present account, the process as defined above makes it possible, in contrast to the conventional granulation processes, to obtain a composition in the form of porous granules with a bulk density μ of less than 0.5 g/cm³.

Another subject-matter of the invention is a film-forming composition as defined above in the form of granules, characterized in that it additionally comprises from 1% to 40% by weight of one or more colouring agents.

The colorants used in the invention are those mentioned in the pharmacopoeias or in the lists of food additives, referenced in Europe under the numbers E 100 to E 172, such as, for example, iron oxides, titanium, zinc or magnesium oxides, colorants absorbed on alumina lakes or alternatively certain natural colorants, such as caramel, carotenoids, riboflavin or chlorophyll. Use may also advantageously be made of composite colorants composed of a combination of potassium aluminium silicates (mica), of titanium dioxide and of colorants, such as those sold by Merck under the name of Candurin™.

According to a specific aspect of the present invention, the film-forming composition as defined above additionally comprises from 1% to 20% by weight of one or more technological additives.

The term “technological additives” denotes more particularly agents which promote adhesion of the film, food or pharmaceutical plasticizers which are liquid at ambient temperature, sweeteners, agents for masking taste and/or flavouring agents for food or pharmaceutical use.

Another subject-matter of the invention is a process for colouring the composition as defined above, characterized in that the composition as defined above in the form of granules is dry mixed with one or more colouring agents.

The term “dry mixing” indicates that the fixing stage of the process which is a subject-matter of the present invention is carried out without addition of water or of solvent, by simple mixing of the starting reactants, that is to say colourless porous granules, colouring agents and, optionally, technological additives, which themselves can be in the solid form or in the form of an organic solution. It is clearly understood that the process as defined above comprises neither a stage of wetting by a binding aqueous solution nor a stage of drying, in contrast to the process disclosed in French Patent Applications FR 2 548 675 and FR 2 660 317.

Entirely unexpectedly, the pigments and other technological additives thus employed are fixed in a stable way in the porous granules of the composition which is a subject-matter of the present invention and the coloured granule thus obtained dissolves much more rapidly in water than those prepared by the granulation processes disclosed in the preceding patents.

To prepare the coloured granule, the colourless porous granule, subsequently referred to as base granule, is placed in a high-performance mixer, such as, for example, a Diosna™ or Lodige™ mixer, into which the dyes, lakes, pigments and, optionally, one or more technological additives of the film-forming composition are introduced, one by one or together. Dry mixing is carried out for a few minutes, a time sufficient for the dyes, lakes or pigments and other components to become fixed in the pores and at the surface of the starting granule.

The final coloured granule obtained exists in the form of more or less spherical agglomerates with a particle size characterized by a percentage by weight passing through a 400 μm sieve of less than 25%, a maximum size of 2000 μm and a mean diameter of approximately 600 μm, preferably between 400 and 800 μm. In a more preferred version of the invention, the final coloured granule exhibits a fraction by weight which passes through a 250 μm sieve of less than 10% and a fraction by weight of between 1000 μm and 2000 μm of less than 20%. Its density is generally greater than or equal to 0.2 and less than 0.5 and preferably greater than or equal to 0.3. Its residual water content is less than 6%. 100 g of granules flow freely in less than 20 seconds, according to the 2-9-16 test described in the European Pharmacopoeia, 4th edition.

Its dispersion in water is evaluated in a standard way in a 2 litre plastic container with a diameter of 15 cm into which 880 g of water are weighed at ambient temperature. Stirring is begun using a deflocculating paddle with a diameter of 8 cm rotating at approximately 500 revolutions per minute. 120 g of coloured granule are then gradually introduced over approximately 1 minute. The progress of the dispersion is assessed by periodically measuring, on withdrawn samples, the amount of solid remaining on a 125 μm sieve. The dispersion is described as rapid if there is no longer any residue remaining on the 125 μm sieve after 15 minutes.

According to a particularly advantageous aspect of the invention, the coloured composition as defined above comprises at least 20% by weight of colouring agents.

Such compositions make possible the preparation of coloured and homogeneous thin films on tablets, sugar-coated tablets or sweets with very slight dry deposits, and thus fast and economically advantageous film coating operations. The following Example 2 describes such an application, which makes it possible to carry out film coating in approximately 25 minutes, whereas conventional coating compositions require dispersing times of the order of 30 to 45 minutes and then film coating times of an additional 30 to 45 minutes more.

In order to obtain a sprayable dispersion, that is to say a dispersion having a viscosity of less than 1000 mPa·s at 25° C., preferably of less than 600 mPa·s, the solids content of the aqueous dispersion is generally between 7% and 60% by weight, preferably between 10% and 30% by weight.

For this reason, another subject-matter of the invention is an aqueous colouring composition, characterized in that it comprises:
from 7% to 60% of a composition as defined above, and
from 40% to 93% of water,
   and the process for the preparation of the aqueous colouring composition as defined above, characterized in that it comprises mixing, in water, one or more coloured compositions as defined above.

The solids content of the aqueous dispersion is adjusted according to the polymers used.

A final subject-matter of the invention is a process for film coating tablets, sugar-coated tablets or sweets, characterized in that it employs the aqueous colouring composition as defined above.

The aqueous dispersion obtained is sprayed over the tablets moving in a perforated rotating pan or in a fluidized bed. A stream of hot air, entering at a temperature generally of greater than or equal to 40° C., provides for the drying of the spray dispersion. In the case of sugar-coated tablets or sweets, film coating has to be carried out with drying air having a temperature preferably between approximately 30° C. and 40° C.

The examples below illustrate the invention and are given without implied limitation.

### EXAMPLE A

### Preparation of a “Base” Granule by Wet Granulation (Comparative Example)

16.8 kg of HPMC 6 cps (Pharmacoat™ 606) and 3.1 kg of microcrystalline cellulose (Vivapur™ 105) are poured into a Diona™ V-100 mixer-granulator. The mixture is homogenized for 5 minutes and then 55 litres of water are gradually added at ambient temperature in order to obtain a wet mass, which is subsequently milled and dried in a fluidized bed until a residual water content of approximately 1% is obtained.

The dry granules obtained are white in colour.

Their bulk density, determined according to the experimental protocol 2-9-15 of the European Pharmacopoeia, is 0.66.

The particle size distribution, obtained by sieving 100 g of granules in a column of standard sieves placed on a “Labomoderne™ S+S” rotary sieving device vibrating at the speed 100 for 10 minutes, is as follows:
9% by weight have a size <315 μm
37% by weight have a size within the range [315 μm, 1000 μm], and
54% by weight have a size within the range [1000 μm, 2000 μm].

Their flow time, determined by the 2-9-16 test described in the European Pharmacopoeia, 4th edition, is less than 10 seconds.

### EXAMPLE 1

### Preparation of Colourless Granules (Example According to the Invention)

12.6 kg of HPMC 6 cps (Pharmacoat™606) and 2.4 kg of microcrystalline cellulose (Vivapur™ 105) are granulated in a Glatt™ GPCG 60 fluidized bed. The mixture of the two powders is first of all homogenized by blowing with air for 6 minutes and then 14 litres of water are gradually sprayed in order to obtain a wet mass, which is subsequently dried by blowing with hot air at 95° C. until a residual water content of approximately 4% is achieved.

The dry granules obtained are white in colour.

Their bulk density, determined according to the experimental protocol 2-9-15 of the European Pharmacopoeia, is equal to 0.22.

The particle size distribution, obtained by the method described in Example A, is as follows:
6% by weight have a size <400 μm,
10% by weight within the range [400 μm, 500 μm],
58% by weight within the range [500 μm, 1000 μm],
26% by weight within the range [1000 μm, 2000 μm].

Its flow time, determined by the 2-9-16 test described in the European Pharmacopoeia, 4th edition, is equal to 21 seconds.

### EXAMPLE 2

### Green Film-Forming Granulated Composition (Example According to the Invention): Preparation and Use

(a)—Preparation

The colourless granules prepared in Example 1 are used to manufacture a green film-forming composition in a single stage by simple dry mixing.

To do this, 1400 g of colourless granules, 534 g of white pigment (titanium dioxide, colorant E 171), 24 g of quinoline yellow, in the alumina lake form (colorant E 104), and 42 g of patent blue V, in the alumina lake form (colorant E 131), are poured into a Diosna™V10 mixer.

After mixing these compounds at speed 1 for 1 minute, green granules are obtained.

Their bulk density, determined according to the experimental protocol 2-9-15 of the European Pharmacopoeia, is equal to 0.39.

The particle size distribution, obtained by the method described in Example A, is as follows:
9% by weight <250 μm,
15% by weight within the range [250 μm, 400 μm],
13% by weight within the range [400 μm, 500 μm],
48% by weight within the range [500 μm, 1000 μm], and
15% by weight within the range [1000 μm, 2000 μm].

The true fixing of the lakes and pigments to the colourless granules is demonstrated by virtue of the following results.

The particle size distribution of the final green granulated film-forming composition is similar to that of the initial colourless composition (see Example 1) In fact, the colouring agents are very fine solid particles with a diameter of less than 100 μm. If they were not intimately incorporated into the colourless porous granule, the particle size distribution of the coloured product obtained would exhibit a level of particles of less than 250 am which would be of the order of 36%.

Each of the particle size fractions obtained is redispersed in water at a solids content of 15% and the trichromatic coordinates L, a, b of each dispersion are measured using a Minolta™ CR 200 chromameter. The following table shows that there is no significant difference in colour between the various fractions, thus demonstrating that the pigments have indeed been homogeneously fixed to all the particles of base granule.

| Fraction | <400 μm | [400-500 μm] | [500-1000 μm] | [1000-2000 μm] |
|---|---|---|---|---|
| L | 68.0 | 68.6 | 67.7 | 67.5 |
| a | −27.3 | −28.1 | −28.0 | −27.6 |
| b | −6.2 | −6.8 | −7.0 | −6.8 |

A keg comprising 5 kg of the green granulated film-forming composition obtained is subjected to vibrations for several days, so as to bring about possible segregation of the fine and dense pigment particles. Samples are then withdrawn at the top, at the middle and at the bottom of the keg and are monitored in order to determine the percentage therein of fines (percentage by weight of powders passing through a 150 μm sieve), in order to assess visually the colour thereof and in order to determine the trichromatic coordinates of an aqueous dispersion comprising 15% by weight of solids. The results recorded in the following table do not reveal any significant difference between the various samples withdrawn, which proves the homogeneity of the contents of the keg and thus the reality of the fixing of the lakes to the colourless granules.

| | | After vibrations | | |
|---|---|---|---|---|
| | | Withdrawal | Withdrawal | Withdrawal |
| | Before | at the top | at the middle | at the bottom |
| | vibrations | of the keg | of the keg | of the keg |
| Fines | 6.5% | 5.1% | 6.9% | 6.1% |
| (% by | | | | |
| weight) | | | | |
| Pantone | 3252C | 3252C | 3252C | 3252C |
| colour | | | | |
| L | 68.0 | 68.2 | 68.5 | 67.7 |
| a | −27.5 | −27.0 | −27.4 | −28.0 |
| b | −6.8 | −6.1 | −6.9 | −7.0 |

(b)—Coating of tablets
(i)—Preparation of the Aqueous Dispersion

75 g of the green granulated film-forming composition prepared in the preceding paragraph are dispersed in 425 g of water at ambient temperature using a deflocculating paddle rotating at 500 revolutions per minute. After 15 minutes, all the green granules are dispersed in the water and the dispersion is used to film coat pharmaceutical tablets with a weight of 550 mg and a diameter of 12 mm.

By way of comparison, the preparation was carried out of an aqueous dispersion of granules with the same chemical composition as those prepared in the preceding paragraph (a) but which were obtained by wet granulation according to the process disclosed in the French patent published under the number FR 2 548 675. The results recorded in the following table reveal that the aqueous dispersion of granules according to the invention no longer comprises any solid residue after stirring for fifteen minutes, whereas that of the state of the art still comprises some after stirring for more than thirty minutes.

TABLE 1
Presence of solid residues on a 125 μm filter in aqueous dispersions of
granules subjected to stirring with a deflocculating paddle

Time (minutes)

| | 5 | 15 | 30 | 45 |
|---|---|---|---|---|
| Granule according to | Yes | None | None | None |
| the invention | | | | |
| Granule according to | Yes | Yes | Slight | None |
| FR 2 548 675 | | | | |

(ii)—Coating of Tablets

The dispersion obtained in the preceding paragraph (i) is sprayed over 1000 g of tablets placed in an Erweka™ coating pan using a Binks™ gun equipped with a spray nozzle with a diameter of 0.8 mm. Spraying as fine droplets is provided by compressed air at 2×10⁵ Pa (2 bar). The spraying flow rate is maintained at 6 g/minute, the drying air temperature is 55° C. and the temperature of the tablets is maintained in the vicinity of 30° C.

Samples are withdrawn after deposition of 0.5%, 0.75%, 1%, 1.25% and 1.5% by weight of solids on the tablets.

Defects of uniformity in colour and in coverage are observed up to a degree of colouring representing the deposition of 0.5% of dry deposit. From 0.75% of deposit, the tablets have a very uniform green colour.

The time necessary for carrying out a homogeneous film coating is therefore reduced to 8 minutes, plus the 15 minutes of time for preparing the dispersion, which represents a saving in time of at least 50% with respect to the film coating carried out under the same conditions with the dispersion with the state of the art.

### EXAMPLE 3

### Preparation of a Red Film-Forming Composition (Example According to the Invention)

The colourless composition prepared according to Example 1 is used to manufacture a red film-forming composition in a single stage by simple dry mixing. 1400 g of colourless granules, 96 g of white pigment (titanium dioxide E 171), 102 g of tartrazine, in the alumina lake form (E 102), 174 g of orange yellow, in the alumina lake form (E 110), and 228 g of erythrosine, in the alumina lake form (E 127), are poured into a Diosna™ V 10 mixer. After mixing these compounds for 1 minute at speed 1, red granules are obtained.

Their bulk density, determined according to the experimental protocol 2-9-15 of the European Pharmacopoeia, is equal to 0.40.

The particle size distribution, obtained by the method described in Example A, is as follows:
6% by mass <250 μm
14% within the range [250 μm, 400 μm],
12% within the range [400 μm, 500 μm],
52% within the range [500 μm, 1000 μm], and
16% within the range [1000 μm, 2000 μm].

This particle size distribution is similar to that of the starting colourless granules, which proves the true fixing of the lakes and pigments to the base granule.

A 15% dispersion in water of this red granulated film-forming composition is subsequently prepared with a deflocculating paddle rotating at 500 revolutions per minute. The dissolution is complete after 15 minutes.

### EXAMPLE 4

### Manufacture of a Coloured Plasticized Film-Forming Composition: Preparation and Use (Example According to the Invention)

The colourless composition prepared according to Example 1 is used to manufacture a red film-forming composition in a single stage by simple dry mixing.

1000 g of colourless granules are poured into a Diosna™ V 10 mixer and 135 g of polyethylene glycol 400, as liquid plasticizer, are gradually added using a Binks™ gun. Mixing is carried out with low-speed stirring in order to satisfactorily distribute the plasticizer without damaging the colourless granule. 135 g of titanium dioxide (E 171) and 260 g of erythrosine lake (E 127) are subsequently added in succession. After mixing these compounds for 1.5 minutes at speed 1, red granules are obtained which flow freely and which have a particle size and a bulk density (μ=0.40) very similar to those given in Example 3.

A 12% dispersion of this granule in water is prepared in approximately 15 minutes with stirring using a deflocculating paddle. It is sprayed, using a Binks™ gun equipped with a 0.8 mm spray nozzle, over 3000 g of tablets placed in a ventilated Driacoater™ pan. Spraying as fine droplets is provided by compressed air at 2×10⁵ Pa (2 bar). The spraying flow rate is maintained at 10 g/minute. The temperature of the entering drying air is 65° C., that of the exiting air is 40° C. and that of the tablets is thus maintained at 33° C. After deposition of 3% of solids, film coated tablets with a very uniform red colour are obtained.

By way of comparison, a dispersion of Sepifilm™ 5938, a product with an identical chemical composition but prepared by wet granulation according to the procedure disclosed in the patent published under the number FR 2 548 675, was prepared under the same operating conditions. The dispersing time necessary for no particle with a diameter of greater than 125 μm to remain is 40 minutes.

The film coating of tablets using this Sepifilm™ 59938 dispersion gives the same result as that obtained with the composition according to the invention but the preparation of the dispersion is markedly slower than with the red composition according to the invention.

### EXAMPLE 5

### Film-Forming Composition for Coating Sweets: Preparation and Use (Example According to the Invention

(a)—Preparation

(i)—Colourless Composition

A colourless composition is prepared by the process described in Example 1 above from the following constituents:
HPMC: 75% by weight
Vivapur™ 105: 11% by weight
Stearic acid: 14% by weight

The bulk density, determined according to the experimental protocol 2-9-15 of the European Pharmacopoeia, is equal to 0.23.

The particle size distribution, obtained by the method described in Example A, is as follows:
2% by weight <400 μm,
10% within the range [400 μm, 500 μm],
78% within the range [500 μm, 1000 μm], and
10% within the range [1000 μm, 2000 μm].
   (ii)—Coloured Composition

1400 g of the colourless granules prepared in the preceding paragraph, 480 g of titanium dioxide (E 171), 22 g of quinoline yellow, in the alumina lake form (E 104), 38 g of patent blue V, in the alumina lake form (E 131), and 60 g of titanium oxide-coated mica (Candurin™ Silver Fine) are poured into a Diosna™ V 10 mixer. After mixing the compounds for 1 minute at speed 1, a green granule, which flows freely, is obtained.

The bulk density, determined according to the experimental protocol 2-9-15 of the European Pharmacopoeia, is equal to 0.37.

The particle size distribution obtained is similar to that of the starting colourless composition.

(b)—Coating of Sweets

A dispersion of 75 g of green granules, obtained in the preceding paragraph, in 425 g of water at ambient temperature is prepared using a deflocculating paddle. The dissolution is assessed by periodically measuring, on withdrawn samples, the amount of solid remaining on a 125 μm sieve. After 15 minutes, all the green granules have dispersed in the water and the resulting dispersion is used to film coat 3000 g of pieces of chewing gum in a ventilated Driacoater™ pan by spraying using a Binks™ gun equipped with a spray nozzle with a diameter of 0.8 mm.

Spraying as fine droplets is provided by compressed air at 2×10⁵ Pa (2 bar). The spraying flow rate is gradually increased over one hour from 1.2 g to 4 g of dispersion per minute. The temperature of the entering air is maintained at approximately 32° C. After 1 hour 40 minutes, 315 g of dispersion have been sprayed, which corresponds to a dry deposit of 1.5% on the pieces of chewing gum. Pieces of chewing gum which are coloured green, which are very glossy and which have a homogeneous and blotch-free colour are obtained.

### EXAMPLE 6

### Brown Film-Forming Composition for Coating Tablets: Preparation and Use (Example According to the Invention)

(a)—Preparation

(i)—Colourless Composition

A colourless composition is prepared in a Glatt GPCG300 industrial air bed by granulating the following composition:
HPMC: 168 kg
Microcrystalline cellulose 31 kg
   using 337 kg of water introduced at a flow rate of the order of 2 kg/min. During the granulation operation, the temperature of the entering air is maintained at 85° C.

A porous granule is finally obtained which exhibits a residual water content of 2% and which has a bulk density, determined according to the experimental protocol 2-9-15 of the European Pharmacopoeia, of equal to 0.25.

The particle size distribution, obtained by the method described in Example A, is as follows:
0.3% by weight <400 μm,
6.9% within the range [400 μm, 500 μm],
72.8% within the range [500 μm, 1000 μm], and
11.4% within the range [1000 μm, 2000 μm].
   (ii)—Coloured Composition

14 000 g of the colourless granules prepared in the preceding paragraph, 5400 g of titanium dioxide (E 171), 480 g of yellow iron oxide (E 171Y) and 120 g of red iron oxide (E 171R) are poured into a Diosna™ V 100 mixer. After dry mixing the compounds, a brown granule which flows freely is obtained.

The bulk density, determined according to the experimental protocol 2-9-15 of the European Pharmacopoeia, is equal to 0.38.

The particle size distribution, obtained by the method described in Example A, is as follows:
5.0% by weight <400 μm,
9.6% within the range [400 μm, 500 μm],
76.8% within the range [500 μm, 1000 μm], and
8.6% within the range [1000 μm, 2000 μm].

It is very similar to that of the starting colourless composition, which again proves that the particles of colouring agent have indeed been incorporated within the colourless porous granules.

(b)—Coating of Tablets

A dispersion of 75 g of brown granules, obtained in the preceding paragraph, in 425 g of water at ambient temperature is prepared using a deflocculating paddle.

The dissolution is assessed by periodically measuring, on withdrawn samples, the amount of solid remaining on a 125 μm sieve. After 15 minutes, all the brown granules have dispersed in the water and the resulting dispersion is used to film coat 3000 g of tablets in a ventilated Driacoater™ pan by spraying using a Binks™ gun equipped with a spray nozzle with a diameter of 0.8 mm.

Spraying as fine droplets is provided by compressed air at 2×10⁵ Pa (2 bar). The spraying flow rate is maintained at 10 g/minute. The temperature of the entering drying air is 65° C., that of the exiting air is 40° C. and that of the tablets is thus maintained at 33° C. After deposition of 3% of solids, film coated tablets with a very uniform brown colour are obtained.

1-18. (canceled)

19. A granular composition which may be used for forming films, wherein:
a) said composition comprises:
1) about 30% to about 100%, by weight, of at least one film forming polymer;
2) about 0% to about 30%, by weight, of at least one plasticizer; and
3) about 0% to about 70%, by weight, of at least one inert filler; and
b) said composition has a bulk density less than about 0.5 g/cm³.

20. The composition of claim 19, wherein the granules of said composition have a non-zero porosity.

21. The composition of claim 19, wherein said film-forming polymer comprises at least one member selected from the group consisting of:
a) hydroxypropylmethyl celluloses;
b) ethyl celluloses;
c) methyl celluloses;
d) carboxymethyl celluloses;
e) hydroxypropyl celluloses;
f) cellulose acetates;
g) phthalates;
h) carrageenans;
i) sodium;
j) potassium alginates;
k) ammonium alginates;
l) modified starches;
m) dextrins;
n) maltodextrins;
o) guar gum;
p) gum tragacanth;
q) gum arabic;
r) xanthan gum;
s) polyvinylpyrrolidones;
t) polyvinyl alcohols; and
u) acrylic polymers.

22. The composition of claim 19, wherein said composition comprises about 50% to about 100%, by weight, of said film-forming polymer.

23. The composition of claim 22, wherein said composition comprises about 50% to about 90%, by weight, of said film forming polymer.

24. The composition of claim 19, wherein said plasticizer comprises at least one member selected from the group consisting of:
a) sorbitol;
b) mannitol;
c) xylitol;
d) glycerol;
e) sucrose;
f) polyethylene glycols;
g) propylene glycol;
h) stearic acid;
i) stearic acid salts;
j) polyethoxylated stearic acid;
k) fatty acid monoglycerides;
l) fatty acid diglycerides
m) fatty acid diglyveride derivatives esterifled by acetic acid;
n) tartaric acid
o) lactic acid;
p) esters of fatty acids and of propylene glycol;
q) esters of fatty acids and of sorbitol;
r) esters of fatty acids and of sorbitan;
s) esters of fatty acids and of mannitol;
t) esters of fatty acids and of mannitan,
u) sucrose esters;
v) sucroglycerides; and
w) polyglycerol esters.

25. The composition of claim 22, wherein said stearic acid salts comprise at least one member selected from the group consisting of:
a) magnesium stearate; and
b) aluminum stearate.

26. The composition of claim 19, wherein said composition comprises about 0% to about 20%, by weight, of said plasticizer.

27. The composition of claim 19, wherein said inert filler comprises at least one member selected from the group consisting of:
a) microcrystalline cellulose;
b) lactose;
c) talc; and
d) silica.

28. The composition of claim 19, wherein said composition comprises about 0% to about 50%, by weight, of said inert filler.

29. The composition of claim 28, wherein said composition comprises about 10% to about 50%, by weight, of said inert filler.

30. The composition of claim 19, further comprising less than about 10%, by weight, of water.

31. The composition of claim 30, wherein said composition comprises less than about 6%, by weight, of said water.

32. The composition of claim 19, wherein said composition has a bulk density of less than about 0.4 g/cm³.

33. The composition of claim 20, wherein:
a) less than about 25%, by weight, of said granules have a particle size less than about 500 μm; and
b) no more than about 15%, by weight, of said granules have a particle size greater than about 2000 μm.

34. A method which may be used for preparing a film forming composition, wherein:
a) the composition has a bulk density less than about 0.5 g/cm³, and wherein said composition comprises:
1) about 30% to about 100%, by weight, of at least one film forming polymer;
2) about 0% to about 30%, by weight, of at least one plasticizer; and
3) about 0% to about 70%, by weight, of at least one inert filler; and
b) said method comprises:
1) homogenizing at least said film forming polymer in a fluidized bed by blowing said polymer with air;
2) forming a wet mass by gradually spraying said polymer with water; and
3) drying said wet mass with hot air.

35. The method of claim 34, further comprising:
a) homogenizing an additional component in a fluidized bed by blowing said additional component with air, wherein said additional component comprises at least one member selected from the group consisting of:
1) said plasticizer; and
2) said inert filler;
b) forming a wet mass by gradually spraying said additional component with water; and
c) drying said wet mass with hot air.

36. The composition of claim 19, further comprising about 1% to about 40%, by weight, of at least one coloring agent.

37. The composition of claim 36, wherein said coloring agent comprises at least one member selected from the group consisting of:
a) iron oxides;
b) titanium;
c) zinc oxides;
d) magnesium oxides;
e) colorants absorbed on alumina lakes; and
f) natural colorants, wherein said natural colorants comprise at least one member selected from the group consisting of:
1) caramel;
2) carotenoids;
3) riboflavin; and
4) chlorophyll.

38. The composition of claim 36, further comprising about 1% to about 20%, by weight, of at least one additive, wherein said additive comprises at least one member selected from the group consisting of:
a) agents which promote adhesion of the film;
b) food plasticizers which are liquid at ambient temperature;
c) pharmaceutical plasticizers which are liquid at ambient temperature;
d) sweeteners;
e) agents for masking taste;
f) food flavoring agents; and
g) pharmaceutical flavoring agents.

39. The method of claim 35, further comprising dry mixing said film forming composition with at least one coloring agent, wherein said coloring agent comprises at least one member selected from the group consisting of:
a) iron oxides;
b) titanium;
c) zinc oxides;
d) magnesium oxides;
e) colorants absorbed on alumina lakes; and
f) natural colorants, wherein said natural colorants comprise at least one member selected from the group consisting of:
1) caramel;
2) carotenoids;
3) riboflavin; and
4) chlorophyll.

40. The method of claim 39, further comprising dry mixing said film forming composition with at least one additive, wherein said additive comprises at least one member selected from the group consisting of:
a) agents which promote adhesion of the film;
b) food plasticizers which are liquid at ambient temperature;
c) pharmaceutical plasticizers which are liquid at ambient temperature;
d) sweeteners;
e) agents for masking taste;
f) food flavoring agents; and
g) pharmaceutical flavoring agents.

41. The composition of claim 36, wherein said composition comprises about 20%, by weight, of said coloring agent.

42. A composition which may be used as an aqueous coloring composition, wherein said composition comprises:
a) about 40% to about 93%, by weight, of water; and
b) about 7% to about 60%, by weight, of a granular component, wherein:
1) said component has a bulk density less than about 0.5 g/cm³; and
2) said component comprises:
i) about 30% to about 100%, by weight, of at least one film forming polymer;
ii) about 0% to about 30%, by weight, of at least one plasticizer;
iii) about 0% to about 70%, by weight, of at least one inert filler; and
iv) about 1% to about 40%, by weight, of at least one coloring agent.

43. The composition of claim 42, wherein:
a) the granules of said component have a non-zero porosity;
b) less than about 25%, by weight, of said granules have a particle size less than about 500 μm; and
c) no more than about 15%, by weight, of said granules have a particle size greater than about 2000 μm.

44. A method which may be used for coating objects comprising forming a film on objects by applying an aqueous coloring composition, wherein
a) said objects comprise at least one member selected from the group consisting of:
1) tablets;
2) sugar-coated tablets; and
3) candy; and
b) said aqueous coloring composition comprises:
1) about 40% to about 93%, by weight, of water; and
2) about 7% to about 60%, by weight, of a granular component, wherein:
i) said component has a bulk density less than about 0.5 g/cm³; and
ii) said component comprises:
aa) about 30% to about 100%, by weight, of at least one film forming polymer;
bb) about 0% to about 30%, by weight, of at least one plasticizer;
cc) about 0% to about 70%, by weight, of at least one inert filler; and
dd) about 1% to about 40%, by weight, of at least one coloring agent.
